(19) United States
(12) Patent Application Publication
(10) Pub. No.: US 2010/0298720 A1
(43) Pub. Date: Nov. 25, 2010

(54) In Situ Energy Harvesting Systems for Implanted Medical Devices

(76) Inventor: Joseph Allen POTKAY, Sheffield Village, OH (US)

Correspondence Address: BioTechnology Law Group;12707 High Bluff Drive, Suite 200, San Diego, CA, 92130-2037, US

(21) Appl. No.: 12/762,242

(22) Filed: Apr. 16, 2010

Related U.S. Application Data
(60) Provisional application No. 61/170,102, filed on Apr. 16, 2009.
(60) Provisional application No. 61/212,999, filed on Apr. 17, 2009.

(51) Int. Cl. A61N 1/378; A61B 5/021; A61B 5/026; A61B 5/01

(52) U.S. Cl. 600/485; 607/35; 607/5; 600/505; 600/549

(57) ABSTRACT

This invention concerns miniature implantable power sources that harvest or scavenge energy from the expansion and contraction of biological tissues, for example, an artery or a bundle of muscle fiber. Such power sources employ an energy harvesting element that converts mechanical or thermal energy existing or generated in or from a pulsatile tissue into a form of electrical energy that can be used or stored by an implanted medical device, such as a blood pressure sensor, a flow meter, or the like. Preferred energy harvesting element embodiments utilize a piezoelectric thin film embedded within a flexible, self-curling medical-grade polymer or coating. Such power sources can be used to produce self-powered implanted microsystems with continuous or near-continuous operation, increased lifetimes, reduced need for surgical replacement, and minimized or eliminated external interface requirements.

### RELATED APPLICATIONS

This patent application claims priority to and the benefit of U.S. provisional patent application Ser. Nos. 61/170,102 and 61/212, 999, filed on 16 and 17 Apr. 2009, respectively, the contents of each of which are hereby incorporated by reference in their entirety for any and all purposes.

### GOVERNMENT RIGHTS

Research related to this invention was supported by Department of Veterans Affairs Rehabilitation Research and Development Grant C3819C, The Advanced Platform Technology Center of Excellence. The U.S. government may have certain rights in this invention.

### TECHNICAL FIELD

This invention concerns devices and systems capable of harvesting energy in situ from biological systems in order to provide power for implanted medical devices.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

Implanted microsystems have the potential to revolutionize health care and dramatically improve health and well-being. Many of such devices are currently being investigated and some, such as the CardioMEMS EndoSure® device (www.cardiomems.com), have received government approval for human implantation. However, a major source of limitation for such devices is their source of power. Batteries enable continuous or periodic operation; however, they require frequent recharging, have finite lifetimes, may be hazardous if fractured, and their replacement requires surgery. Wirelessly powered devices, on the other hand, remove the battery (and its drawbacks) from the system, but require an external interface for operation. Such interfaces can be damaged, are burdensome to carry, and cosmetically unappealing. In addition, frequent periodic measurements are inconvenient at best.

Energy harvesting devices provide an attractive alternative to wireless and battery power. Ambient sources of energy provide an almost limitless reservoir of energy that can be harvested as needed. Thus, a device that harvests energy from an ambient source would potentially be able to provide continuous power to implanted microsystems without the limitations of batteries (required recharging, limited lifetime). However, sources of ambient energy are limited for implanted microsystems; there is minimal light penetration deep into the body, no appreciable temperature gradients exist below the skin, and movement and vibration are not guaranteed.

Recently, devices that scavenge energy directly from the human body have been investigated. In 1980, Ko, et al. presented a piezoelectric device that harvested energy from the mechanical motion of a beating heart. See W. H. Ko, “Power sources for implant telemetry and stimulation systems,” in A Handbook on Biotelemetry and Radio Tracking, C. J. Amlaner and D. MacDonald, Eds. Elmsford, N.Y.: Pergamon Press, Inc., 1980, pp. 225-245. The 10-cm³ device was surgically connected to the heart and a cantilever and piezoelectric material were utilized to convert mechanical motion into power. The device generated 30 μW when implanted in a dog, but its performance degraded over time due to the attachment of connective tissue. In 1988, Hausler, et al. (“Implantable physiological power supply with PVDF film,” in Medical Applications of Piezoelectric Polymers, P. M. Galletti and D. E. De Rossi, Eds. New York, N.Y.: Gordon and Breach Science Publishers, 1988, pp. 259-264) reportedly utilized a rolled polyvinylidene fluoride (PVDF) film to convert energy from breathing into electrical power. The device, connected between adjacent ribs in a canine, produced 17 μW of continuous power. However, the device would require long implanted electrical leads (and additional surgery) to distribute the power to other parts of the body.

In 2007, Lewandowski et al. investigated a piezoelectric device that was surgically attached between a muscle and tendon and scavenged energy from the expansion/contraction of the muscle. A 2-cm³ version of this device was predicted to generate 690 μW of power when attached to the gastrocnemius muscle. See B. E. Lewandowski, K. L. Kilgore and K. J. Gustafson, “Design considerations for an implantable, muscle powered piezoelectric system for generating electrical power,” Ann. Biomed. Eng., vol. 35, pp. 631-641, 2007. Such a device, however, is dependent on contraction of a muscle and, thus, power generation is neither constant nor guaranteed; such properties in a power supply would be dangerous and risky in implantable systems designed for detection and early warning of hazardous conditions. To date devices such as those discussed above have only been applicable in very limited regions of the body and/or have provided intermittent power that is not guaranteed. These and other shortcomings largely limit their applicability for real world application in conjunction with implantable medical devices.

In contrast to conventional approaches, the present invention concerns devices that harvest, or scavenge, energy from a biological source that is both continuous and available throughout the body, reliable, and can readily be adapted for use with a wide variety of implantable devices and systems. Energy from blood pressure variations would meet the criteria above; it is both continuous and widely available throughout the body. The remainder of the paper presents work on an arterial cuff energy scavenging (ACES) device that, for the first time, converts the expansion and contraction of an artery (due to changes in blood pressure) into electrical energy for use in implanted microsystems.

### DEFINITIONS

When used in this specification, the following terms will be defined as provided below unless otherwise stated. All other terminology used herein will be defined with respect to its usage in the particular art to which it pertains unless otherwise noted.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

In the context of chemicals (e.g., carbon dioxide, various hydrocarbons, oxides of nitrogen, etc.), the term “species” refers to a population of chemically indistinct molecules of the sort referred to, i.e., is a population of small molecules identified by the same chemical formula.

### SUMMARY OF THE INVENTION

The energy harvesting devices of the invention allow for the development of autonomous, integrated, self-powered implantable microsystems capable of providing for improved monitoring and treatment of conditions and diseases such as heart failure, high-level spinal cord injury, and aneurysms.

Thus, one aspect of the invention concerns patentable in situ biological energy harvesting devices. These devices harvest or recover a portion of the energy inherent in biological systems, such as living animals, including humans. In particular, they harvest mechanical and/or thermal energy present inside living organisms and convert it to electrical energy that can then be used for other desired purposes, for example, to power one or more implantable medical devices intended to monitor one or more physiological parameters inside a patient and/or to deliver a therapy, such as cardiac pacing, cardiac defibrillation, or drug (e.g., a hormone such as insulin, a chemotherapeutic agent, etc.). Such power sources can be used to produce self-powered implanted microsystems with continuous or near-continuous operation, increased lifetimes, reduced need for surgical replacement, and minimized or eliminated external interface requirements.

The energy harvesting devices of the invention preferably include an energy harvesting element, for example, a piezoelectric thin film, disposed in a resilient biocompatible insulator, preferably a medical-grade polymer or coating. The resilient biocompatible insulator ensures that the device will be suitable for long-term placement in a patient; that it is resilient means that it can, for example, repeatedly expand and contract with appreciable degradation in its expansion and contraction function over the intended useful life of the device. Preferred configurations include cuffs and sleeves adapted for energy-transferring association with a pulsatile tissue, for example, a blood vessel such as an artery, a bundle of skeletal muscle fibers, or a blood vessel graft (e.g., and arterial graft). Particularly preferred embodiments resilient self-curling medical-grade silicone cuffs sized for energy-transferring association with an artery or arterial graft.

A related aspect of the invention relates to implantable medical devices that utilize one or more of the instant energy harvesting devices as a power supply. Such devices or microsystems include cardiac stimulation devices (e.g., pacemakers, defibrillators, cardioverters, etc.), neurostimulators, and drug pumps. Other such microsystems include those for monitoring or sensing one or physiological parameters in a patient. In addition to an energy harvesting device according to the invention, such the instant microsystems also include such circuitry and data processing, analysis, and storage hardware and software, and other components as is necessary to perform the intended function(s).

Another aspect of the invention concern methods of using an energy harvesting device according to the invention to harvest or recover energy generated in a patient and convert it so that it can be used to power an implantable medical device. Still other aspects of the invention concern methods for making and using such energy harvesting devices, as well as implantable medical devices that include such energy harvesting devices as a power source.

Other features and advantages of the invention will be apparent from the following drawings, detailed description, and appended claims.

### BRIEF DESCRIPTION OF THE FIGURES

FIG. 1 is an illustration of an autonomous, implantable self-powered blood pressure sensing system that contains and integrated energy harvesting device according to the invention.

FIG. 2 has two illustrations. FIG. 2(a) is a diagram showing three views of a self-curling energy harvesting element in its resting position (left) and before curling (right; side and top views). FIG. 2(b) illustrates a mechanical model for the determination of the strain in the piezoelectric. K_{PIEZO} is the mechanical spring constant of the piezoelectric film, K_{sIL,A} is the mechanical spring constant of the silicone elastomer in parallel with the piezoelectric, and K_{SIL,B} is the mechanical spring constant of the silicone elastomer in the area of the cuff without the embedded piezoelectric material.

FIG. 3 shows three plots showing the theoretical impact of the piezoelectric (PVDF) dimensions on the power output of an energy harvesting element according to the invention. All other dimensions are held constant during the simulations.

FIGS. 4 and 5 are photographs of an energy harvester according to the invention in its resting state (FIG. 4) and wrapped around a mock artery in the test setup (FIG. 5).

FIG. 6 shows two graphs. FIG. 6(a) is a graph showing measured power output from an energy harvesting element as described in Example 1, below. The graph in FIG. 6(b) is an expanded view of a five second timeframe of the measured operation of the device described in Example 1, below. The non-optimized device generates an average power of 6 nW when a simulated blood pressure (top waveform) is applied to the mock artery.

FIG. 7 shows a block diagram of an integrated, implantable autonomous self-powered blood pressure monitoring microsystem, as described in Example 2, below.

FIG. 8(a) shows a circuit topology used to model an autonomous implantable microsystem. FIG. 8(b) is a graph showing a simulation of the circuit of FIG. 8(a) using a supply voltage generated by an energy harvester as described in Example 1, below.

FIG. 9 shows a process flow for making a microfabricated integrated microsystem comprising and arterial energy harvester integrated with a blood pressure/strain sensor, as is described in Example 4.

FIG. 10 is a diagram of the blood pressure/strain sensor microfabricated into the microsystem of FIG. 9 as a series of interdigitated gold electrodes that, when stretched as a result of arterial expansion, for example, increase in capacitance.

FIG. 11 shows schematics of different AC-to-DC conversion circuitry described in Example 4. Replacement of a diode-connected MOSFET as shown to the left of the arrow with a circuit as shown on the right can reduce turn-on voltage from |V_{TP}|(˜0.72 V) to |V_{TP}|−V_{TN}(˜0.17 V) and increase the efficiency of energy harvesting. D₁ and D₂ are diode-connected MOSFETs.

### DETAILED DESCRIPTION

As those in the art will appreciate, the following detailed description describes certain preferred embodiments of the invention in detail, and is thus only representative and does not depict the actual scope of the invention. Before describing the present invention in detail, it is understood that the invention is not limited to the particular aspects and embodiments described, as these may vary. It is also to be understood that the terminology used herein is for the purpose of describing particular embodiments only, and is not intended to limit the scope of the invention defined by the appended claims.

### 1. Introduction.

The present invention relates to in situ energy harvesting devices and systems that can be used to power autonomous, self-powered implanted microsystems. With the advancements in microfabrication technology driven by Moore's Law, these microsystems are no longer limited by the sensors and circuits comprising the sensing and data processing components of such microsystems, but rather by their power sources. Batteries, fuel cells, and microengines have finite lifetimes and are potentially hazardous, and their replacement requires costly surgeries with increased risk of complications. While wireless inductive powering techniques avoid at least some of these difficulties, they require an external interface for operation. Miniature or small size is also critical in order to minimize the implanted microsystem's invasiveness once placed in a patient, typically by a surgical procedure, preferably a minimally invasive surgical procedure.

Sources of ambient or in situ energy provide an attractive alternative to fixed energy sources. Whereas fixed energy sources (e.g., batteries, fuel cells, etc.) require recharging and/or periodic replacement, ambient biological sources provide an almost limitless reservoir of energy that can be harvested as needed. Preferred are biological sources of energy are those that are constant and available at various locations throughout a patient's body, guaranteeing an easily accessible power supply necessary for continuous long-term autonomous operation after the device is implanted.

Energy from blood pressure variations meets these criteria, particularly energy from arterial blood pressure variations (or pressure variations that occur in other tissues that can give rise to changes in diameter or circumference of a volume of such tissue, e.g., a bundle of muscle fibers), as it is both continuous and widely available throughout the body. As is known, a typical adult blood pressure waveform has systolic/diastolic pressures of 155/80 mmHg at a pulse of 60 beats per minute (bpm); however, blood pressure can range from 250/150 mmHg in very severe hypertension to 50/30 mmHg in extreme hypotension. In addition, heart rates can vary from 45 to over 200 bpm. During variations in blood pressure, an artery's diameter expands and contracts. The diameter of the distal abdominal aorta in adult Caucasian males, for example, varies between 15.8 mm and 17.3 mm as a result of variations in blood pressure associated with normal heart function. In the process of expanding and contracting following each ventricular contraction, in the process, the distal abdominal aorta (and other arteries throughout the body) converts variations in pressure into variations in mechanical strain. Accordingly, a primary focus of the invention is to harvest ambient biological energy from a pulsatile tissue or material, such as an artery or arterial graft (including arterial grafts made from biocompatible synthetic materials and veins), bundles of muscles fibers, and the like.

### 2. Energy Harvesting Devices.

The energy-harvesting element can take any form now known or later developed that can be adapted to convert mechanical or thermal energy existing or generated in or from a pulsatile tissue (e.g., an artery, bundle of muscle fiber, etc.) into a form of electrical energy that can be used or stored. Combinations of such elements can also be used. For example, certain preferred embodiments on the invention concern energy-harvesting elements that can convert variations of blood pressure inside an artery or arterial graft, or the expansion/contraction of the artery/graft due to those blood pressure variations, into electrical energy. Such elements include, without limitation, one or a combination of the following:

1) A piezoelectric or electroactive/electrostrictive polymer (see, e.g., Y. Liu, K. L. Ren, H. F. Hofmann, Q. Zhang, “Investigation of electrostrictive polymers for energy harvesting,” IEEE Transactions on Ultrasonics, Ferroelectrics and Frequency Control, Vol. 52, No. 12, 2005, pp. 2411-2417; F. Carpi, D. De Rossi, “Electroactive polymer-based devices for e-textiles in biomedicine,” IEEE Transactions on Information Technology in Biomedicine, Vol. 9, No. 3, 2005, pp. 295-318) disposed in a one or more layers as an energy-harvesting element capable of converting mechanical strain experienced by the layer due to expansion and/or contraction of the artery or graft into electrical energy. The layer may be directly strained by the expansion/contraction of the artery/graft or may be indirectly strained through the use of, for example, a proof mass and beam that deflect with each expansion/contraction of the blood vessel (or graft).

2) An electrostatic/capacitive energy-harvesting element that converts a change of capacitance into electrical energy (J. A. Paradiso and T. Starner, “Energy scavenging for mobile and wireless electronics,” IEEE Pervasive Computing, vol. 4, pp. 18-27, 2005; Beeby, S. P., Tudor, M. J. and White, N. M. (2006) Energy harvesting vibration sources for microsystems applications. Measurement Science and Technology, 17 (12). R175-R195). In such embodiments, the capacitor can be formed such that the stretching of the cuff stretches the dimensions of the capacitor, changing its value. Alternatively, the capacitor can be formed from the plates a cavity-based pressure sensor [citation(s)?]. In this case, the expansion/contraction of the artery and associated changes in pressure will alter the distance between the plates of the capacitor, changing its value.

3) An electromagnetic harvester in which the expansion/contraction of, for example, an artery causes a magnet to move relative to a coil generating power. In one such embodiment, the magnet is positioned on one side of the cuff and the coil is placed on the other side. As the artery expands and contracts, the distance between the two changes, an electromagnetic field (EMF) is induced in the coil, and power is generated.

4) A temperature-based thermoelectric or pyroelectric energy harvesting element (C. Watkins, B. Shen and R. Venkatasubramanian, “Low-grade-heat energy harvesting using superlattice thermoelectrics for applications in implantable medical devices and sensors,” in 24th International Conference on Thermoelectrics (ICT '05), 2005, pp. 265-267; A. Cuadras, M. Gasulla, A. Ghisla, V. Ferrari, “Energy Harvesting from PZT Pyroelectric Cells,” Instrumentation and Proceedings of the IEEE Measurement Technology Conference, 2006, pp. 1668-1672) that scavenges energy due to the difference in temperature between the artery/graft and the region external to the artery/graft. In addition, the temperature of the surrounding environment may vary as the artery/graft expands and contracts. This change can also be used to would generate electrical power in such an element.

5) A turbine-based energy harvester in which the movement of a fluid, caused by the expansion/contraction of the artery, generates a pressure difference between the blades of the turbine. Such a pressure difference will cause the blades of the turbine to rotate and this rotation can be converted to useful power. The fluid movement can be caused by the natural movement of fluids external to the graft or by an integrated fluidic channel located within the energy-harvesting element.

Preferred energy-harvesting elements include those made from piezoelectric materials.

Energy-harvesting elements piezoelectric materials suitable for use in practicing the invention can be purchased or fabricated. For example, Measurement Specialties Inc. manufactures films comprised of piezoelectric materials (e.g., PDVF) down to 9 μm in thickness. Microfabrication techniques can be used to produce films having any desired dimensional characteristics from any suitable piezoelectric material (or combinations thereof).

Energy harvesting elements are typically disposed in a biocompatible insulating material, along with the electrical leads and connectors necessary to make the desired electrical connections to other circuitry or devices.

The energy harvesting elements of the invention are typically configured as devices that provide for energy-transferring association with the pulsatile tissue with which they will be associated in vivo. The particular configuration will vary depending upon the particular application, and will allow mechanical and/or thermal energy to be transferred from the pulsatile tissue to the energy-harvesting element(s) of the device. For example, in certain embodiments where energy is to be harvested from the expansion and contraction of an artery, the energy harvesting element(s) is(are) preferably disposed in a flexible, resilient biocompatible insulator adapted to provide for energy transfer from the artery to the energy-harvesting element(s). Configurations such as cuffs or sleeves suitable for placement about the pulsatile tissue, here, an artery, are preferably employed. The cuff or sleeve may completely or partially surround or encircle the blood vessel. Spiraling configurations can also be employed.

### 3. Integrated Microsystems.

The energy harvesting devices of the invention are incorporated into integrated, preferably autonomous, implantable medical devices and/or sensors. Alternatively, an energy harvesting device according device according to the invention can be implanted remotely from the implantable medical device(s) and/or sensor(s) for which it provides energy. In such embodiments, energy is transferred from the energy harvesting device to an implantable medical device via any suitable electrical connection, including cables, wires, and electrical leads configured for implantation. Energy from the energy harvesting device can be used to directly power the implantable medical device and/or alternatively to charge an energy storage device (e.g., one or more batteries, a capacitor, etc.) or system that powers the implantable medical(s) device and/or sensor(s).

Representative medical devices that can be powered using an in situ energy harvesting device according to the invention cardiac stimulation devices, cardiac or other physiological monitoring devices (e.g., blood pressure and/or flow sensors), neurostimulators, implantable drug pumps, and the like. Such devices may be programmable, including programming via an external device. Such devices or systems may also include telemetry capability.

Implantable cardiac stimulation devices are well known, and include implantable defibrillators and cardioverters to treat accelerated rhythms of the heart such as fibrillation as well as implantable pacemakers to maintain the heart rate above a prescribed limit, such as, for example, to treat a bradycardia. Implantable cardiac stimulation devices that incorporate both a pacemaker and a defibrillator are also known.

In general, a pacemaker includes two major components, a pulse generator to generate pacing stimulation pulses and the lead, or leads, having electrodes to electrically couple the pacemaker to the heart. These devices (and other implantable medical devices that employ one or more energy harvesting units according to the invention) also include electronic circuitry and a power source (e.g., one or more batteries). Such devices can provide for unipolar and bipolar pacing. See, e.g., U.S. Pat. No. 7,676,265.

Pacemakers deliver pacing pulses to the heart to induce a depolarization and a mechanical contraction of that chamber when the patient's own intrinsic rhythm fails. To this end, pacemakers include sensing circuits that sense cardiac activity for the detection of intrinsic cardiac events such as intrinsic atrial events (P waves) and intrinsic ventricular events (R waves). By monitoring such P waves and/or R waves, the pacemaker circuitry is able to determine the intrinsic rhythm of the heart and provide stimulation pacing pulses that force atrial and/or ventricular depolarizations at appropriate times in the cardiac cycle when required to help stabilize the electrical rhythm of the heart.

As the foregoing makes clear, devices of the invention also include those configured to monitor or sense one or more physiological parameters in a patient in whom the device is implanted. Any desired physiological parameter, or set of physiological parameters, can be sensed, provided that the appropriate sensor(s) or monitoring component(s) is(are) incorporated within or is otherwise functionally associated with the device. Physiological parameters that can be sensed include blood pressure, fluid flow rate, temperature, electrical activity (e.g., cardiac P waves, R waves, QRS complexes, brain wave activity, etc.) in a tissue or organ (e.g., the heart, skeletal muscle, smooth muscle, the brain, etc.), oxygen content, drug or hormone level, and/or any other medically relevant physiological parameter.

### 4. Applications.

The instant energy harvesting devices and autonomous, implantable integrated microsystems incorporating them have numerous applications, particularly in disease treatment and monitoring. In this regard, cardiovascular diseases (CVDs), such as coronary heart disease and congestive heart failure, currently affect approximately 80 million U.S. patients. More than one third of all deaths in the U.S. in 2004 were due to CVD. In addition, approximately 1.5 million people in the U.S. are living with abdominal aortic aneurysms (AAA), and 200,000 new cases occur annually. AAA's are the 13th leading cause of death in this country. For the first time, an autonomous implantable microsystems of the invention, once implanted in a CVD patient, will allow for completely autonomous periodic monitoring of these and other conditions, which will lead to improved monitoring, advanced detection of complications, reduced mortality rates, and improved quality of life for patients with such ailments. In addition, and as those in the art will appreciate, the blood pressure-based energy harvesting, energy storage, and low-power measurement technologies embodied in such microsystems can readily be adapted for use in conjunction with many other implantable devices designed to treat and/or monitor a wide variety of other diseases and conditions.

### EXAMPLES

The following Examples are provided to illustrate certain aspects of the present invention and to aid those of skill in the art in practicing the invention. These Examples are in no way to be considered to limit the scope of the invention in any manner.

### Example 1

### An Arterial Cuff Energy Harvester for Implanted Medical Microsystems

This example describes a miniature implantable power source that harvests (or scavenges) energy from the expansion and contraction of a mock artery. The energy harvesting element of the 0.25 cm³ device utilizes a piezoelectric thin film embedded within a flexible, self-curling medical-grade silicone cuff. Such an element can enable self-powered implanted microsystems with near-continuous operation, increased lifetime, reduced surgical replacement, and minimized or eliminated external interface requirements compared to conventional implanted medical devices. The fabricated device described in this example generates up to 16 nW when tested around a mock artery. Microfabricated versions of such an energy harvesting element should be capable of generating power outputs of greater than 1.0 μW.

### I. INTRODUCTION

The example describes an arterial cuff energy scavenging (ACES) device that, for the first time, converts the expansion and contraction of an artery (due to changes in blood pressure) into electrical energy that can be used to power implanted microsystems, including implanted medical devices.

### II. OVERVIEW

A typical blood pressure waveform has systolic/diastolic pressures of 115/80 mmHg and a pulse of 60 beats per minute (bpm). However, blood pressure can range from 250/150 mmHg in very severe hypertension to 50/30 mmHg in extreme hypotension. In addition, heart rates can vary from 45 to over 200 bpm. An artery's diameter expands and contracts with variations in blood pressure. The diameter of the distal abdominal aorta in adult Caucasian males, for example, varies between 15.8 mm and 17.3 mm for a blood pressure of 118/64 mmHg and a heart rate of 66 bpm (T. Lanne, H. Stale, H. Bengtsson, D. Gustafsson, D. Bergqvist, B. Sonesson, H. Lecerof and P. Dahl, “Noninvasive Measurement of Diameter Changes in the Distal Abdominal Aorta in Man,” Ultrasound in Medicine, vol. 18, pp. 451-458, 1992). Using these values, the power expended by blood pressure (to cause the artery to expand) over a 1-cm length of the abdominal aorta can be calculated to be 2.5 mW.

A device that harvests energy from blood pressure is safe. That is, it should not occlude or hamper blood flow or restrict arterial movement significantly. In addition, it minimizes or eliminates the risks of infections, blood clotting, and of stroke or heart attack. The device is also located outside the arterial wall due, unlike intra-arterial devices that, by virtue of their placement, necessarily interact directly with blood (increasing the risk of complications) and may dislodge causing a stroke. Finally, the device is miniature in size in order to minimize its impact on the body and have a long lifetime to reduce the need for surgical replacement.

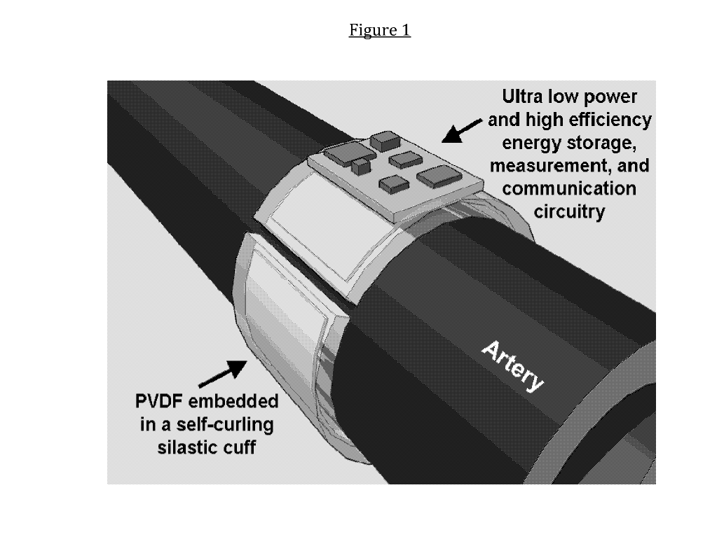

The arterial cuff energy scavenging (ACES) device shown in FIG. 1 meets all of the requirements. In the figure, the energy harvesting element is shown integrated into a self-powered blood pressure monitoring cuff. The energy harvesting element of the ACES device is enclosed, included within, or coated a thin, flexible sheath of biocompatible insulation (for example, a medical grade silicone elastomer, e.g., Silastic) that is designed to be self-curling. Nerve electrodes with a similar self-curling mechanical structure have been previously shown to be capable of expanding and contracting to fit closely to the surface of a nerve without compressing the nerve or damaging it. G. G. Naples, J. T. Mortimer, A. Scheiner and J. D. Sweeney, “A spiral nerve cuff electrode for peripheral nerve stimulation,” IEEE Trans. Biomed. Eng., vol. 35, pp. 905-916, 1988; J. D. Sweeney, D. A. Ksienski and J. T. Mortimer, “A nerve cuff technique for selective excitation of peripheral nerve trunk regions,” IEEE Trans. Biomed. Eng., vol. 37, pp. 706-715, 1990; W. M. Grill and J. T. Mortimer, “Neural and connective tissue response to long-term implantation of multiple contact nerve cuff electrodes,” J. Biomed. Mater. Res., vol. 50, pp. 215, 2000.

The energy harvesting element of the ACES device comprises a thin piezoelectric film is integrated into the biocompatible insulation and converts the expansion/contraction of the artery into electrical energy. Piezoelectric energy conversion has the advantage that it does not require a power source or complex circuitry and can produce output voltages similar to that required for measurement circuitry; the same is not true of capacitive and inductive conversion methods. Finally, polyvinylidene fluoride (PVDF) film was chosen over other piezoelectric materials due to its low Young's modulus (˜3 MPa), lack of hazardous materials (such as lead in PZT), and its ability to be formed in very thin sheets (<30 μm).

### III. THEORY

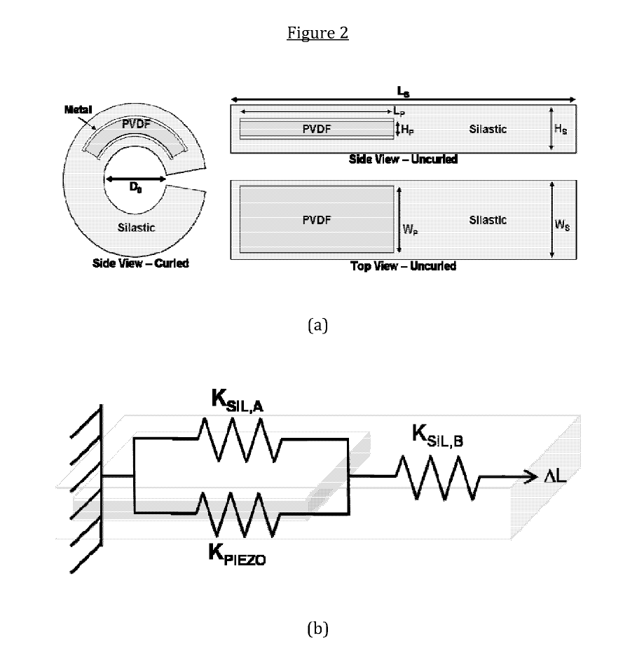

A first order mechanical/electrical model was developed to predict the device's behavior and optimize its performance. This model makes the following assumptions: 1) arterial diameter varies linearly with pressure; 2) the blood pressure waveform is sinusoidal; 3) the mechanical properties of the ACES are dominated by the medical grade silicone-based insulation Silastic; and 4) the ACES is a closed cylinder. In this model, blood pressure causes expansion of the artery, artery expansion creates strain in the arterial wall and ACES device, and strain is converted into power by the piezoelectric energy harvesting element. Each one of these conversions has been modeled utilizing an analytical relationship as described below. Relevant device dimensions are shown in FIG. 2(a).

The artery wall and ACES are both modeled as open-ended thick-walled cylinders in order to predict the expansion/contraction of the artery after the ACES is attached. Equation (1) gives the relationship between pressure and diameter for an open-ended thick-walled cylinder (Naples, et al, supra).

$$Δ P = \frac{E ⌊ {( {r}_{i} + t )}^{2} - {r}_{i}^{2} ⌋}{2 {r}_{i}^{3} [ ( 1 + v ) + ( 1 + v ) \frac{{( {r}_{i} + t )}^{2}}{{r}_{i}^{2}} ]} Δ D \qquad ( 1 )$$

In Equation 1, ΔP is the change in blood pressure, ΔD is the change in diameter, E is the elastic modulus of the material, v is the Poisson's ratio, rᵢ is its initial radius, and t is the thickness of the layer. This equation is used for the arterial wall and ACES to determine the overall relationship between blood pressure and arterial diameter. The “spring constant”, ΔP/ΔD, of each layer is calculated and added together to determine the total “spring constant”, which is then used to predict the change in diameter of the arterial wall. For simplicity, the cuff is assumed to be dominated by the silicone; this assumption holds true for PVDF lengths that are appreciably shorter than the length of the silicone in the cuff, preferably less than about 20-75% the length of the silicone in the cuff.

Arterial wall expansion stretches the cuff, generating strain in the cuff. In other words, the change in diameter of the artery stretches the cuff, generating strain that is split between the silicone and PVDF components of the energy harvesting element. In order to determine the strain in the PVDF, each component was modeled as a linear mechanical spring. See FIG. 2(b). The strain in the piezoelectric layer was then utilized to calculate the voltage and power generated by the PVDF, which was modeled in the electrical domain as a strain-dependent voltage source in series with a capacitor. Equations (2)-(5), below give the voltage generated by the PVDF, the capacitance of the material, the optimal load resistance, and the instantaneous power delivered to an optimal load resistor, respectively.

V_{PIEZO}=g₃₁X₁H_{P} (2)

$$\begin{matrix}{C}_{PIEZO} = \frac{{ɛ}_{r} {ɛ}_{0} {W}_{P} {L}_{P}}{{H}_{P}} & ( 3 ) \\ {R}_{LOAD} = \frac{1}{2 π \cdot f \cdot {C}_{PIEZO}} & ( 4 ) \\ P = \frac{{V}_{PIEZO}^{2}}{4 {R}_{LOAD}} = \frac{π}{2} {g}_{21}^{1} {X}_{1}^{2} f {ɛ}_{r} {ɛ}_{0} {W}_{P} {L}_{P} {H}_{P} & ( 5 )\end{matrix}$$

In Equations (2)-(5), g₃₁ is the piezoelectric stress coefficient, X₁ is stress applied to the PDVF film, f is the frequency of application, εᵣ is the relative permittivity of the material, ε₀ is the permittivity of free space, and W_{P}, L_{P}, and H_{P}, are the width, length, and height of the PVDF film, as shown in FIG. 2.

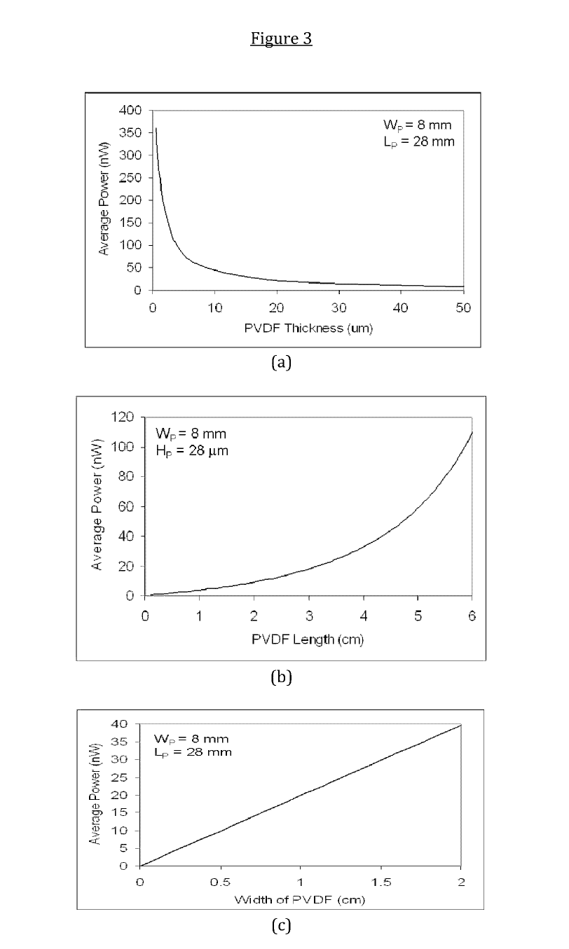

By combining all aspects of the theoretical model, the relationship between the device's dimensions and its predicted performance can be determined. FIG. 3 shows the theoretical impact that the PVDF thickness, length, and width have on the device's average power output into an optimal resistive load. The default piezoelectric dimensions for each plot are W_{P}=8 mm, L_{P}=28 mm, and H_{P}=28 μm. FIG. 3(a) displays the effect that varying the PVDF thickness has on device performance. As the thickness decreases, the PVDF becomes less stiff and experiences more strain relative to the silicone, increasing power output and decreasing constriction of the artery. Thus, the PVDF thickness should be minimized as much as possible. FIG. 3(b) displays the impact of PVDF length on the power output of the ACES device. As the length increases, the strain in the PDVF relative to that in the silicone increases, increasing the power output. However, as the length increases, the PVDF begins to dominate the mechanical properties of the device and it restricts arterial expansion. It should also be noted that as the PVDF length becomes comparable to the total length of the cuff, one of the model's assumptions becomes invalid reducing its accuracy. FIG. 3(c) displays the linear effect that varying the PVDF width has on device performance.

Overall, the fabricated prototype described in this example having dimensions W_{P}=8 mm, L_{P}=28 mm, and H_{P}=28 μm is predicted to generate an average power of 16 nW into an optimal resistive load (Eq. 4). If the width and length of the PVDF are increased to 15 mm and 40 mm, respectively, and the thickness is decreased to 1 μm, the power output is predicted to increase to greater than 1.0 μW.

### IV. FABRICATION

The instant PDVF energy-harvesting element converts the deflection of a mock artery due to variations in blood pressure into electrical power. This element includes a piezoelectric thin film embedded within a self-curling sheath of biocompatible insulation. The energy-harvesting element was designed to naturally curl and conform to the shape of an artery or vein without appreciably restricting the vessel's pulsatile capacity (i.e., ability to expand and contract in response to pressure changes of the fluid within the vessel). Energy-harvesting elements that have lengths longer than the circumference of the targeted artery will result in a cuff that wraps around the artery and overlaps itself. Elements of different dimensions can be produced in order to accommodate different artery sizes, differing energy requirements for the microsystems to be powered, etc. As with other energy harvesting devices of the invention, PDVF-based devices are configured such that they provide for energy-transferring association with the pulsatile tissue such that mechanical (or, in other embodiment, thermal energy) from the expansion and contraction of the tissue (e.g., an artery) is transferred to the energy-harvesting element(s) of the device.

Fabrication of the device was accomplished in a class 100 cleanroom. Metallized PVDF from Measurement Specialties Inc. was used as the piezoelectric material due to its mechanical flexibility, piezoelectric properties, and lack of hazardous materials. Electrical contacts (pre-packaged in medical grade silicone) were compression bonded to two flat platinum electrodes and then attached to each side of the PVDF utilizing conductive epoxy. If necessary, PDVF was polled in order to activate its piezoelectric properties.

Next, the PVDF and contacts were embedded in two layers of medical-grade silicone sheeting, which was bonded together utilizing medical-grade epoxy and cured. In order to facilitate curling of the cuff, the top layer of silicone was stretched and remained in tension during curing. After release, the differential stress caused the cuff to curl. The resting diameter of the self-curling cuff was theoretically predicted (see Naples, et al., supra) and perfected through experience. The cuff diameters were designed to be slightly less than the minimum diameter of the targeted artery so that it fit around the artery but did not restrict its expansion.

### V. RESULTS

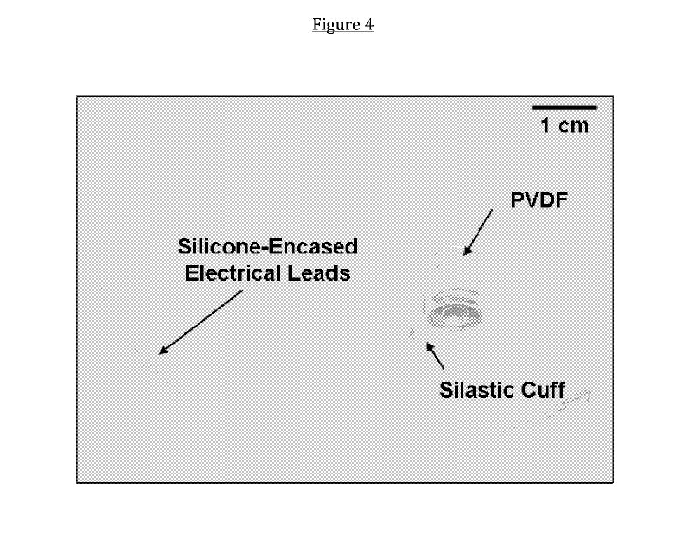

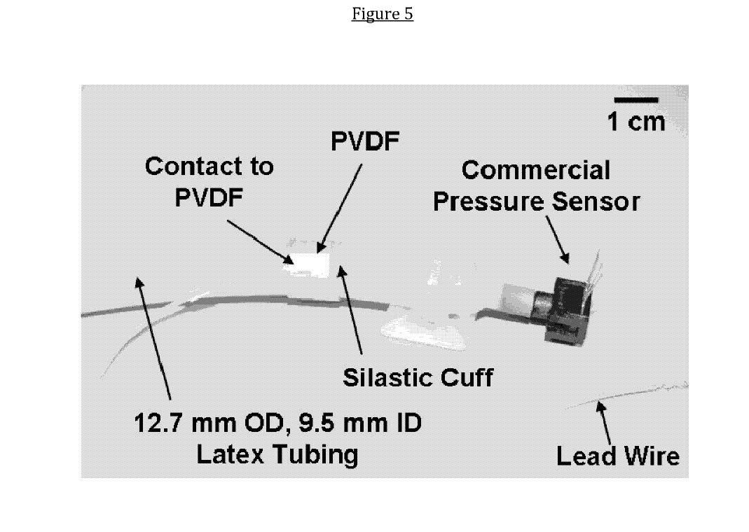

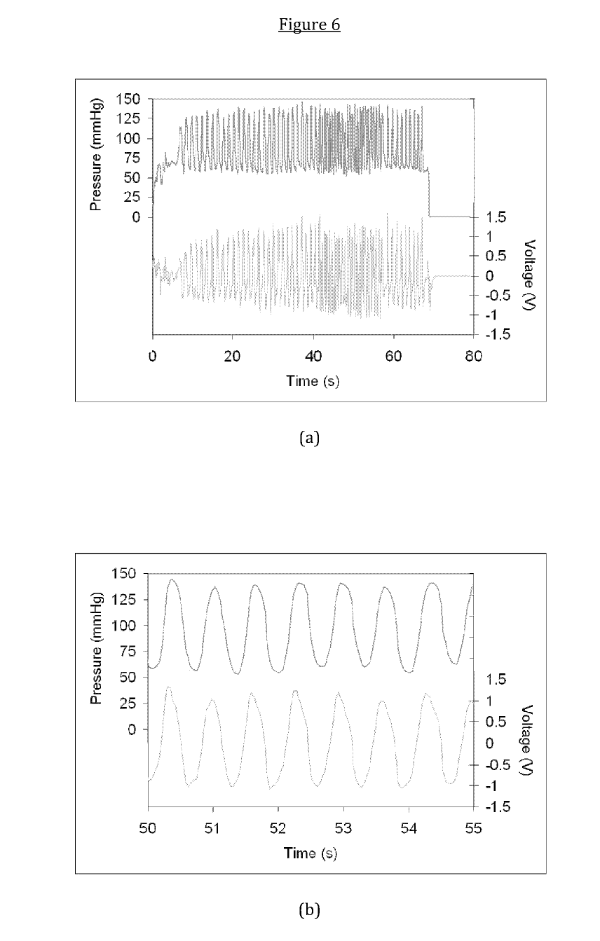

Utilizing the fabrication process above, a prototype device was created consisting of a 28 mm×8 mm×28 μm PVDF thin film embedded inside the 0.25 cm³ self-curling silicone cuff, as shown in FIG. 4. The completed device was tested utilizing the test setup depicted in FIG. 5. Latex tubing (12.7 mm outer diameter, 9.5 mm inner diameter) was used to simulate an artery. The cuff was placed around the mock artery. The capacitance of the PVDF was measured using an LCR meter and found to be 1400 pF; the optimal load resistance was calculated to be 114 MΩ (see Equation 4, above). The PVDF was loaded with an optimal load resistor (Eq. 4) and connected to a high input impedance voltmeter. The pressure inside the tubing was monitored using a commercial pressure sensor. A large distance from the ACES, the tubing was compressed and relaxed in order to generate a time-varying pressure waveform that simulated changes in blood pressure; this pressure was measured with the commercial pressure sensor. FIG. 6 shows the measured output voltage of the arterial cuff energy scavenger. The data in FIG. 6(b) displays a peak voltage of 1.2 V, a maximum instantaneous power of 16 nW and an average power of 6 nW. See Table 1, below.

TABLE I
COMPARISON OF THE THEORETICAL AND
MEASURED PERFORMANCE OF ACES DEVICE

| Parameter | | Theory | Measured |
|---|---|---|---|
| R_{LOAD} | (MW) | 125 | 75 |
| V_{LOAD} | (V) | 2.8 | 1.2 |
| P_{MAX} | (nW) | 32 | 16 |
| P_{AvG} | (nW) | 16 | 6 |

Table I, above, summarizes the theoretical and experimental results for the ACES device made and tested as described in this example. The difference between the theoretical and actual values of R_{LOAD} is due to the parasitic capacitances and actual values of R_{LOAD} are due to the parasitic capacitances of the electrical leads. The difference in the output voltage and power can be attributed to two factors. First, the model approximates the device as an open-ended, thick-walled cylinder, when in fact the device is not a closed cylinder. A more advanced mechanical model is expected to improve the agreement between theoretical and measured data. Second, the model assumes that the pressure waveform is sinusoidal. As can be seen in FIG. 6, this approximation is not exact and causes error in the theoretical model.

### VI. CONCLUSION

This example describes a particularly preferred embodiment of an in situ energy generator, including the theory underlying the device, its fabrication, and experimental results for the device when used under laboratory conditions to convert the expansion and contraction of a simulated artery into electrical power that can be used with any of a variety of implanted microsystems. The ACES device described in this example has a volume of about 0.25 cm³ and generates a peak power of 16 nW when tested on a simulated artery. A microfabricated version of this device, as described above, should generate greater than 1.0 μW.

### Example 2

### An Autonomous, Self-Powered Implanted Medical Microsystem

This example describes an autonomous implantable microsystem having an integrated energy harvesting device according to the invention. This microsystem device integrates an arterial cuff energy-harvesting device as described in Example 1, above, into a blood pressure sensing system with energy storage, measurement, and data storage circuitry (see FIG. 1). Blood pressure sensing is accomplished using a capacitive strain sensor utilizing a varying gap distance. The circuitry necessary to make the self-powered system will utilize low-turn-on-voltage diodes and low-leakage capacitors to rectify and store the electrical signal generated by the energy harvester. This stored energy will then be utilized by the device's blood pressure sensing system to periodically monitor blood pressure within the artery about which the implantable microsystem is deployed.

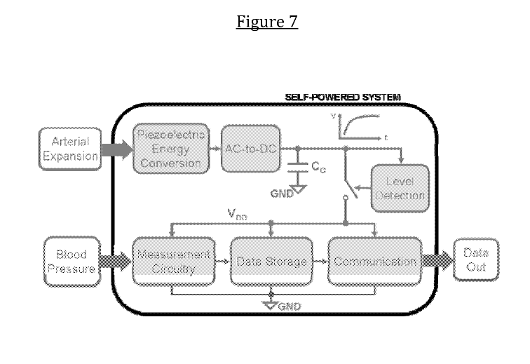

A block diagram of the complete the implantable microsystem is shown in FIG. 7. Expansion and contraction of the artery wall due to variations in blood pressure will be converted into an electrical voltage by the polyvinylidene fluoride (PVDF) film embedded within the biocompatible Silastic cuff. Microelectronic circuitry is then utilized to convert the output voltage of the PVDF into a DC signal that is stored on a large capacitor (C_{C}). This voltage increases over time as the heart beats and artery pulsates. When the stored energy reaches a suitable level, it serves as the power source for the measurement, data storage, and communication circuitry. Level detection circuitry associated with the capacitor determines when there is enough energy available to take a measurement. If the power generated by the PVDF film is sufficient, measurements will be taken at a suitably rapid data acquisition or sampling rate. If less power is available than that needed to, in effect, provide continuous monitoring, measurements will be taken periodically based on the availability of stored energy sufficient to perform the particular measurement. In other words, in this type of device the time between measurements is a function of the power generated by the PVDF, the efficiency of the energy storage circuitry, and the power consumption of the measurement, data storage, and communication circuitry.

Autonomous implantable microsystems such as described in this example will enable completely autonomous periodic monitoring of these and other conditions, which will lead to improved monitoring, advanced detection of complications, reduced mortality rates, and improved quality of life for patients with such ailments. In addition, and as those in the art will appreciate, the blood pressure-based energy harvesting, energy storage, and low-power measurement technologies embodied in such microsystems can readily be adapted for use in conjunction with many other implantable devices designed to treat and/or monitor a wide variety of other diseases and conditions.

### Example 3

### Autonomous Implantable Microsystem Model

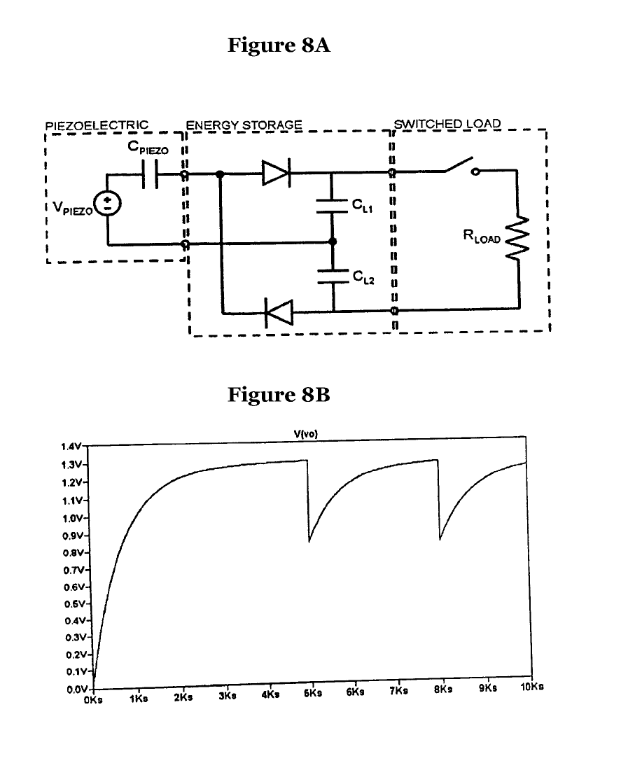

This example describes a representative circuit topology (see FIG. 8(a)) that can be used to simulate or test energy harvesting devices according to the invention. In this circuit the energy harvesting element (e.g., one comprised of a PVDF strip) is modeled as a voltage source in series with a capacitor. The energy storage circuitry utilizes a voltage doubler topology with two storage capacitors. The load is modeled as a resistor that is periodically switched on to draw power from the storage capacitors. As the artery expands and contracts, voltage generated by the PVDF film is rectified and charges up the load capacitors, C_{L1} and C_{L2}. The results of a simulation of the circuit using values from the device described above in Example 1 are shown in FIG. 8(b). In this simulation, approximately 50 minutes was required between measurement cycles. The dips in the voltage represent two simulated measurement cycles that consume 2 μW of power for 1 s. When the measurements are complete, the supply voltage begins to regenerate.

As will be appreciated, such a model can be developed for many different energy harvesters. The specifics of a particular model, and of the corresponding energy harvesting devices and implanted autonomous microsystems powered by such devices, will depend on many factors, including the design and power output of the particular energy harvester, the type of sensor(s) employed for data acquisition, the desired data acquisition rate, the energy storage system(s) employed, the power requirements of the processing, data storage, and data transmission units, etc.

### Example 4

### Autonomous Implantable Blood Pressure Sensor Microsystem

As shown in FIG. 3, the power generating capacity of a piezoelectric-based energy-harvesting device according to the invention increases dramatically as the thickness of the piezoelectric material decreases and as its length increases (increases in width of the material only contribute linear increases to energy-generating capacity). If only the length of the energy harvester is increased, the piezoelectric material begins to dominate the mechanical properties of the device and limit its performance (and potentially constrict the elastic properties of the artery, graft, or tissue about which the device is positioned). However, if the thickness of the piezoelectric material is additionally reduced, large performance gains in terms of power generating capacity can be realized with minimal drawbacks. For this reason, microfabrication techniques that allow for the production of very thin (e.g., from about 0.01 um to about 30 um) energy-harvesting elements can be used to produce various integrated microsystems that include a sensing device powered by an energy harvesting system according to the invention to provide for autonomous power generation following the microsystem's implantation in a patient.

This example describes such an integrated microsystem, namely a fully microfabricated autonomous (i.e., energy-harvesting) blood pressure-monitoring microsystem having the specifications set forth in Table 2.

TABLE 2
Autonomous blood pressure-monitoring microsystem

| Total | | Average Power | Energy | | |
|---|---|---|---|---|---|
| Device | Piezoelectric | Output of | Harvesting | Total Power | Time Between |
| Volume | Dimensions | Piezoelectric | Efficiency | Consumption of | Measurements |
| (cm³) | (L × W × H)(mm) | (mW) | (%) | Circuitry (mW) | (min) |
| 0.25 | 40 × 15 × 0.001 | 1 | 20 | 10 | 2 |

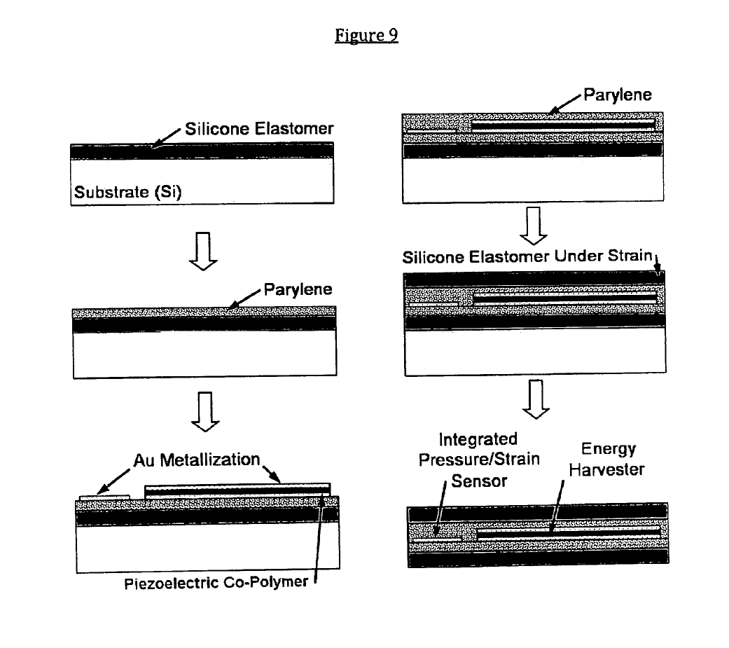

To manufacture this blood pressure-monitoring microsystem, the following microfabrication approach is utilized to produce a capacitance-based blood pressure sensor integrated with a piezoelectric P(VDF-TrFE) co-polymer energy harvester to provide autonomous power. The fabrication sequence is illustrated in FIG. 9. As shown in FIG. 9, silicon wafer is used for structural support and handling during the integrated microsystem's fabrication. Biomedical-grade silicone elastomer (Silastic® MDX4-4210) encapsulates the device, which is patterned using a SU-8 mold. In this process, a silicone elastomer is deposited on a silicon wafer by spin coating and curing. A layer of parylene is then deposited, patterned, and utilized as a moisture barrier. Metallization with gold (or other electrically conductive materials such as titanium, platinum, etc.) is performed using a suitable method to provide a first electrically conductive layer of desired thickness, followed by the addition of a layer of piezoelectric material (e.g., P(VDF-TrFE) co-polymer) having a desired thickness. A second electrically conductive layer (e.g., gold) is then applied on top of the piezoelectric material. Deposition and patterning of the metal-piezoelectric-metal layers form the integrated pressure/strain sensor and energy harvester. Gold metallization can be performed, for example, via sputtering to form the top and bottom electrodes of the piezoelectric polymer-based energy harvesting element. See FIG. 9. P(VDF-TrFE) is deposited via spin-casting. P(VDF-TrFE) is utilized due to its ease of processing in the context of microfabrication, and it similar piezoelectric properties when compared to PVDF. The P(VDF-TrFE) co-polymer is polled in order to activate its piezoelectric properties. Then, another layer of parylene is deposited. Finally, a top layer of silicone is stretched and bonded to the structure and then the complete structure is released from the carrier wafer. The stretched top layer of silicone causes the device to curl upon release.

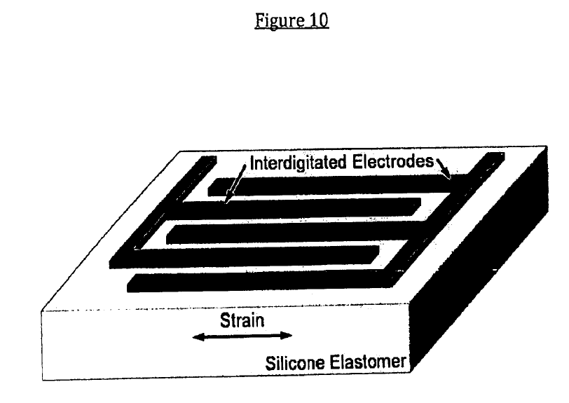

A diagram of the microsystem's interdigitated electrode-based blood pressure sensor is shown in FIG. 10. When the integrated microsystem is positioned about an artery so that, for example, the artery expands with increasing blood pressure, strain is generated in the blood pressure sensor (in addition to the energy-harvester) of the microsystem, which increases the length of the sensor's electrodes, leading to an increase in capacitance. This change in capacitance is correlated to blood pressure. As will be appreciated, other blood pressure sensing structures can also be adapted for use in such microsystems, such as sensors whose electrode overlap varies with arterial expansion. However, capacitive sensors are preferred due to their low power consumption. Finally, the system can include a commercial MEMS pressure sensor to measure pressure external to the cuff and artery (or graft or other tissue). Such a feature will be highly beneficial in measuring, for example, the intra-sac pressure of an aneurysm repaired using an endovascular stent.

As mentioned above, the period of time between measurements by the system is a function of the power generation of the energy harvester, the efficiency of the energy storage circuitry, and the power consumption of the measurement circuitry. Thus, an optimized energy harvester as described above should coupled with high-efficiency energy storage and ultra-low-power measurement and data storage circuitry. An overview of a representative example of such circuitry and the integrated microsystem is shown in FIG. 7. In this example the pulsatile nature of arterial expansion and contraction results in a low-frequency ambient biological energy source from which energy can be harvested by an energy harvester according to the invention in order to provide autonomous power (i.e., power derived from an energy harvester according to the invention that harvests or converts ambient power available from a biological environment in which the device is implanted). The microsystem also includes complex capacitive sensing circuitry and on-board non-volatile data storage that need not be transmitted to an external device after every measurement. All of this circuitry can be implemented on a single chip using a TSMC 0.35 μm 2P/4M n-well standard CMOS process. All circuit components are designed to operate over a wide range of supply voltages (from about 1.8 to 2.3 V, for example) due to the fact that, as configured, the supply voltage decreases as the capacitor discharges. The integrated circuit and any necessary off-chip components can be mounted on a miniature 1-cm² or smaller printed-circuit board that can then be connected with the microsystem and attached thereto with silicone epoxy.

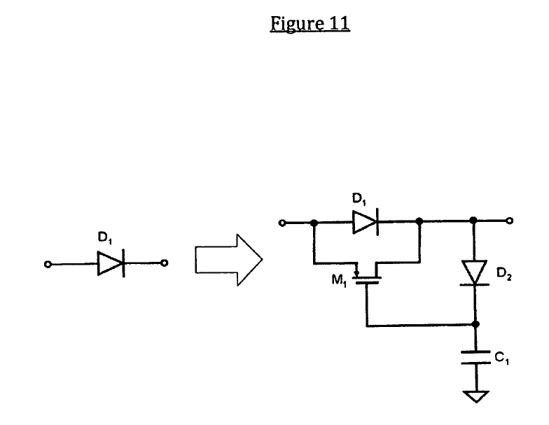

The energy storage system in the microsystem of this example includes an AC-to-DC converter, a storage capacitor, level detection circuitry, and an electronic switch to energize the rest of the system when enough energy has been harvested to provide sufficient power to complete a full operational cycle (i.e., blood pressure measurement, data storage, and, if called for in the particular cycle, data transmission). All of these components are preferably optimized to achieve high energy-harvesting efficiency, minimal power loss, and very low power leakage. For example, using an AC-to-DC converter allows improvement of a voltage doubler such as is shown in FIG. 8. In that circuit the voltage transferred from the piezoelectric (i.e., the energy harvesting element) to the storage capacitors is reduced by the voltage drop of the diodes, reducing the conversion efficiency. Referring now to the improvement shown in FIG. 11, the rectification diodes have been replaced by equivalent circuits with reduced turn-on voltages. Of course, other topologies, including half-wave and full-wave rectifiers and voltage triplers, may also be utilized.

Due to the large required value of the storage capacitor (˜10 μF), it is preferably implemented as an off-chip component. Commercially available, miniature capacitors (e.g., from Hitachi) exhibiting a length and width of about 4 mm and 3 mm, respectively, provide the required capacitance and will still fit into the overall implantable microsystem form factor. Due to the relatively long interval between measurements (perhaps minutes), the storage capacitor should exhibit very low leakage. Suitable level detection circuitry is employed, but it should be optimized for lower power operation. The electronic switch can be implemented as a properly sized PMOS transistor due to its higher threshold voltage and, thus, lower leakage current, compared to NMOS transistors. Overall, an energy-harvesting efficiency greater than 20% will be achieved.

The measurement circuitry will interface with the capacitive pressure sensor to acquire measurements and store them in non-volatile memory; its power consumption directly impacts the period between measurements. In order to minimize the time between measurements, the measurement and data storage circuitry should have a total power consumption less than 10 μW. For the measurement circuitry, capacitance-to-frequency converters are attractive due to the fact that a digital signal can be obtained without an analog-to-digital converter and with minimal circuit components, which features help to minimize power requirements. A relaxation oscillator topology is used due to its low power consumption, lack of required off-chip components, and ability to be designed to be supply insensitive. Blood pressure measurements should have range of −20 to 300 mmHg, a sampling rate of 200 Hz, and an accuracy of 2 mmHg. Thus, an oscillation frequency of approximately 50 kHz will give the required 8 bits of resolution per sample. With an oscillation frequency of 2 MHz and a power consumption of 3 μW, an oscillator operating at 50 kHz should consume significantly less than 3 μW.

Finally, measurement data will be maintained during the periods when the power supply is recharging. A non-volatile flash memory that minimizes power required during a write operation. In order to minimize this power, Folwer-Nordheim tunneling is utilized to charge the floating gate, avoiding the high current required for other techniques such as hot electron injection. The system is optimized for low-voltage, low-power write operations to achieve total microsystem power consumption less than 10 μW.

Although the invention has been described with reference to the above examples, it will be understood that modifications and variations are encompassed within the spirit and scope of the invention. Accordingly, the invention is limited only by the appended claims.

All of the devices, machines, systems, compositions, and methods described and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the spirit and scope of the invention as defined by the appended claims.

All patents, patent applications, and publications mentioned in the specification are indicative of the levels of those of ordinary skill in the art to which the invention pertains. All patents, patent applications, and publications, including those to which priority or another benefit is claimed, are herein incorporated by reference in their entirety to the same extent as if each individual publication was specifically and individually indicated to be incorporated by reference.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

What is claimed is:

1. An in situ biological energy harvesting device comprising an energy harvesting element disposed in a resilient biocompatible insulator and configured as a cuff adapted for energy-transferring association with a pulsatile tissue.

2. An in situ biological energy harvesting device according to claim 1 wherein the pulsatile tissue is a blood vessel, optionally an artery or arterial graft.

3. An in situ biological energy harvesting device according to claim 1 wherein the energy harvesting element comprises a piezoelectric thin film.

4. An in situ biological energy harvesting device according to claim 3 wherein the piezoelectric thin film is embedded in a resilient cuff, optionally a self-curling medical-grade silicone cuff.

5. An in situ biological energy harvesting device according to claim 1 configured as a power source for an implantable medical device, optionally a cardiac stimulation device, a neurostimulator, an implantable drug pump, and device for monitoring or sensing a physiological parameter.

6. An in situ biological energy harvesting device according to claim 1 wherein the implantable medical device is a cardiac stimulation device selected from the group consisting of a pacemaker, a defibrillator, a cardioverter, and a device that includes two or more thereof.

7. An in situ biological energy harvesting device according to claim 1 wherein the implantable medical device that monitors a physiological parameter selected from the group consisting of blood pressure, fluid flow rate, temperature, and electrical activity is a tissue or organ.

8. An implantable medical device that comprises an in situ biological energy harvesting device according to claim 1.

9. A method for monitoring a physiological parameter in vivo, comprising implanting in a patient an implantable medical device according to claim 8 configured to monitor a physiological parameter.

10. A method of treatment, comprising a implanting in a patient an implantable medical device according to claim 8 configured to effect a desired treatment, wherein the implantable medical device optionally is selected from the group consisting of a cardiac stimulation device, a neurostimulator, and a drug pump.
